(19) United States
(12) Patent Application Publication
(10) Pub. No.: US 2016/0263339 A1
(43) Pub. Date: Sep. 15, 2016

(54) CONVERTIBLE PATIENT INTERFACE DEVICE

(71) Applicant: KONINKLIJKE PHILIPS N.V., EINDHOVEN (NL)

(72) Inventor: JOSHUA MARK GREENBERG, ASPINWALL, PA (US)

(21) Appl. No.: 15/032,064

(22) PCT Filed: Oct. 28, 2014
(86) PCT No.: PCT/IB2014/065653

§ 371 Date: Apr. 26, 2016

Related U.S. Application Data
(60) Provisional application No. 61/896,845, filed on Oct. 29, 2013.

(51) Int. Cl. A61M 16/06; A61M 16/08; A61M 16/00

(52) U.S. Cl. CPC A61M 16/0622; A61M 16/0057; A61M 16/0816; A61M 16/0875; A61M 2016/0661

(57) ABSTRACT

A patient interface device includes a fluid coupling member structured to be coupled to a source of breathing gas, and a patient sealing assembly structured to be coupled to the fluid coupling member. The patient sealing assembly includes a central cushion portion, a first side portion and a second side portion, wherein the central cushion portion is structured to enable a size of the central cushion portion to be adjusted in a direction that is generally perpendicular to a longitudinal axis of the patient sealing assembly from a first size configured to provide a nasal/oral mask configured to cover a nose and mouth of a patient to a second size configured to provide a nasal mask configured to cover only the nose of the patient.

### CROSS-REFERENCE TO RELATED APPLICATIONS

This patent application claims the priority benefit under 35 U.S.C. §119(e) of U.S. Provisional Application No. 61/896,845 filed on Oct. 29, 2013, the contents of which are herein incorporated by reference.

### BACKGROUND OF THE INVENTION

1. Field of the Invention

The present invention pertains to patient interface devices structured to deliver a flow of breathing gas to a user, and, in particular, to a patient interface device that may be converted from a nasal/oral mask to a nasal mask and back as desired.

2. Description of the Related Art

There are numerous situations where it is necessary or desirable to deliver a flow of breathing gas non-invasively to the airway of a patient, i.e., without intubating the patient or surgically inserting a tracheal tube into the patient's esophagus. For example, it is known to ventilate a patient using a technique known as non-invasive ventilation. It is also known to deliver positive airway pressure (PAP) therapy to treat certain medical disorders, the most notable of which is OSA. Known PAP therapies include continuous positive airway pressure (CPAP), wherein a constant positive pressure is provided to the airway of the patient in order to splint open the patient's airway, and variable airway pressure, wherein the pressure provided to the airway of the patient is varied with the patient's respiratory cycle. Such therapies are typically provided to the patient at night while the patient is sleeping.

Non-invasive ventilation and pressure support therapies as just described involve the placement of a patient interface device including a mask component having a soft, flexible sealing cushion on the face of a patient. The mask component may be, without limitation, a nasal mask that covers the patient's nose or a nasal/oral mask that covers the patient's nose and mouth, among other configurations. Such patient interface devices may also employ other patient contacting components, such as forehead supports, cheek pads and chin pads. The sealing cushion typically has a support portion coupled to a sealing portion, which may integrated together as a single part or that may be separate components that when combined together in the final assembly provide the sealing and support functions. The patient interface device is connected to a gas delivery tube or conduit and interfaces the ventilator or pressure support device with the airway of the patient, so that a flow of breathing gas can be delivered from the pressure/flow generating device to the airway of the patient. It is known to maintain such devices on the face of a wearer by a headgear having one or more straps adapted to fit over/around the patient's head.

Current patient interface devices employ a single type of mask component. For example, they may employ a nasal/oral mask or a nasal mask. It would be advantageous, however, to have a patent interface device that employs a mask component that may be converted from a nasal/oral mask to a nasal mask and back as desired to suit the needs of the patient.

### SUMMARY OF THE INVENTION

In one embodiment, a patient interface device is provided that includes a fluid coupling member structured to be coupled to a source of breathing gas, and a patient sealing assembly structured to be coupled to the fluid coupling member. The patient sealing assembly includes a central cushion portion, a first side portion and a second side portion, wherein the central cushion portion is structured to enable a size of the central cushion portion to be adjusted in a direction that is generally perpendicular to a longitudinal axis of the patient sealing assembly from a first size configured to provide a nasal/oral mask configured to cover a nose and mouth of a patient to a second size configured to provide a nasal mask configured to cover only the nose of the patient.

In another embodiment, a method of adjusting a patient interface device is provided that includes providing a patient sealing assembly including a central cushion portion, a first side portion and a second side portion, and adjusting a size of the central cushion portion in a direction that is generally perpendicular to a longitudinal axis of the patient sealing assembly from a first size configured to provide a nasal/oral mask configured to cover a nose and mouth of a patient to a second size configured to provide a nasal mask configured to cover only the nose of the patient or from the second size to the first size.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIGS. 1, 2, 4 and 5 are a schematic diagrams of a system adapted to provide a regimen of respiratory therapy to a patient according to one exemplary embodiment;

FIG. 3 is a rear elevational view of a patient sealing assembly forming a part of the system of FIGS. 1, 2, 4 and 5;

FIGS. 6, 7, 8 and 9 are a schematic diagrams of a system adapted to provide a regimen of respiratory therapy to a patient according to an alternative exemplary embodiment; and

FIGS. 10 and 11 are rear elevational views of a patient sealing assembly forming a part of the system of FIGS. 6, 7, 8 and 9.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

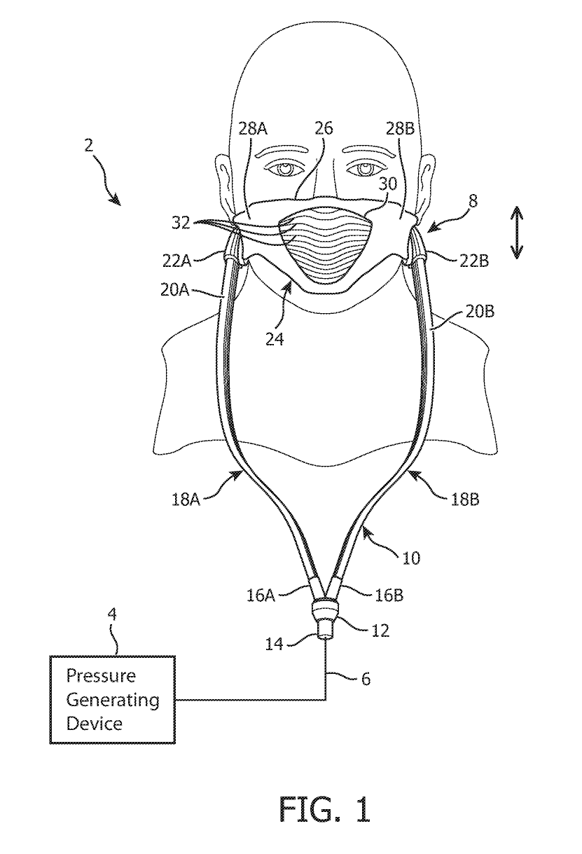

A system 2 adapted to provide a regimen of respiratory therapy to a patient according to one exemplary embodiment is generally shown in FIGS. 1 (front view) and 2 (side view). System 2 includes a pressure generating device 4, a delivery conduit 6, and a patient interface device 8 having a fluid coupling member 10 (which are described in greater detail herein). Pressure generating device 4 is structured to generate a flow of breathing gas and may include, without limitation, ventilators, constant pressure support devices (such as a continuous positive airway pressure device, or CPAP device), variable pressure devices (e.g., BiPAP®, Bi-Flex®, or C-Flex™ devices manufactured and distributed by Philips Respironics of Murrysville, Pennsylvania), and auto-titration pressure support devices. Delivery conduit 6 is structured to communicate the flow of breathing gas from pressure generating device 4 to patient interface device 8 through fluid coupling member 10. Delivery conduit 6 and patient interface device 8 are often collectively referred to as a patient circuit.

In the exemplary embodiment, fluid coupling member 10 includes a one-to-two fluid coupling device 12 having an input port 14 and two output ports 16A, 16B. Fluid coupling member 10 further includes a first mask delivery member 18A coupled to output port 16A and a second mask delivery member 18B coupled to output port 16B. First mask delivery member 18A includes a conduit member 20A having a mask coupling member 22A attached to the distal end thereof. Mask coupling member 22A is structured to fluidly couple conduit member 20A to a first side of patient sealing assembly 24 (described in detail below) via and orifice provided therein. Second mask delivery member 18B includes a conduit member 20B having a mask coupling member 22B attached to the distal end thereof. Mask coupling member 22B is structured to fluidly couple conduit member 20B to a second side of patient sealing assembly 24 via and orifice provided therein.

In addition, in the exemplary embodiment, patient interface device 8 includes a patient sealing assembly 24 which facilitates the delivery of the flow of breathing gas from pressure generating device 4, delivery conduit 6 and fluid coupling member 10 to the airway of a patient. Patient sealing assembly 24 includes a central cushion portion 26, first and second side portions 28A and 28B, and rear portions 30 that are structured to extend around the rear of the patient's head/neck when patient interface device 8 is donned by the patient for securing patient interface device 8 to the head of the patient. Rear portions 30 may include any type of suitable attachment means for attaching to one another, such as, without limitation, a hook and loop fastening system or a magnetic fastening system.

In the exemplary embodiment, patient sealing assembly 24 is made of flexible, cushiony, elastomeric material, such as, without limitation, silicone, an appropriately soft thermoplastic elastomer, a closed cell foam, or any combination of such materials. Also in the exemplary embodiment, at least central cushion portion 26 and first and second side portions 28A and 28B are hollow/inflatable such that the flow of breathing gas can be received in first and second side portions 28A and 28B from first and second mask delivery members 18A and 18B, and then delivered to central cushion portion 26 for subsequent delivery to the airways of the patient.

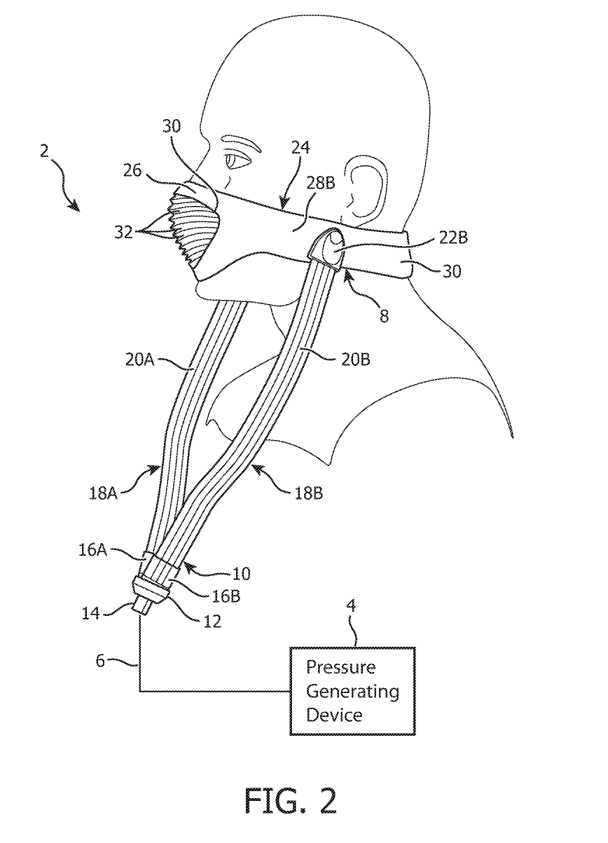

As seen in FIGS. 1 and 2, central cushion portion 26 has an adjustable section 30 having generally horizontal bellows, accordion or convoluted surface features 32 that can fold outwardly or inwardly to allow the size of central cushion portion 26 to be selectively adjusted in a direction that is generally perpendicular to the longitudinal axis of patient sealing assembly 24 as shown by the arrows in FIG. 1.

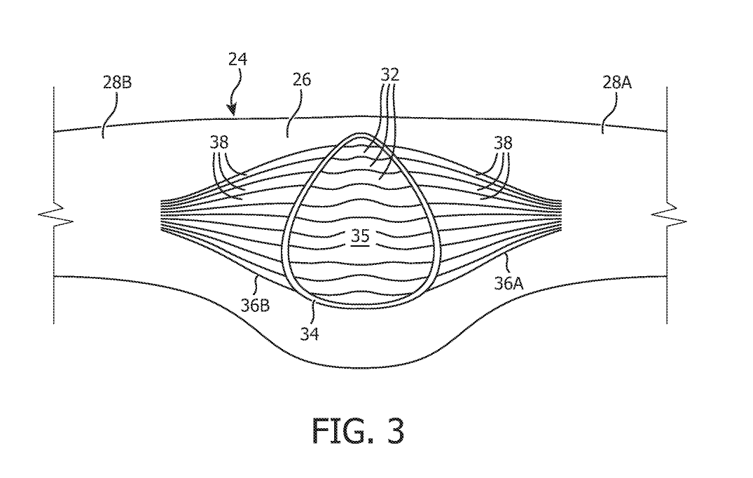

FIG. 3 is a partial rear elevational view of patient sealing assembly 24. As seen in FIG. 3, the rear side of central cushion portion 26 of patient sealing assembly 24 further includes a generally triangularly shaped sealing surface 34 defining a fluid delivery opening 35 and first and second adjustable section 36A and 36B. First and second adjustable sections 36A and 36B each have generally horizontal bellows, accordion or convoluted surface features 38 that can fold outwardly or inwardly to facilitate the adjustment of central cushion portion 26 as described above. In the configuration shown in FIG. 3, sealing surface 34 is sized and structured to sealingly surround the nose and mouth of the patient to allow patient sealing assembly 24 to function as a nasal/oral mask component. As described below, the size of sealing surface 34 may be adjusted to sealingly surround just the nose of the patient to allow patient sealing assembly 24 to function as a nasal mask component.

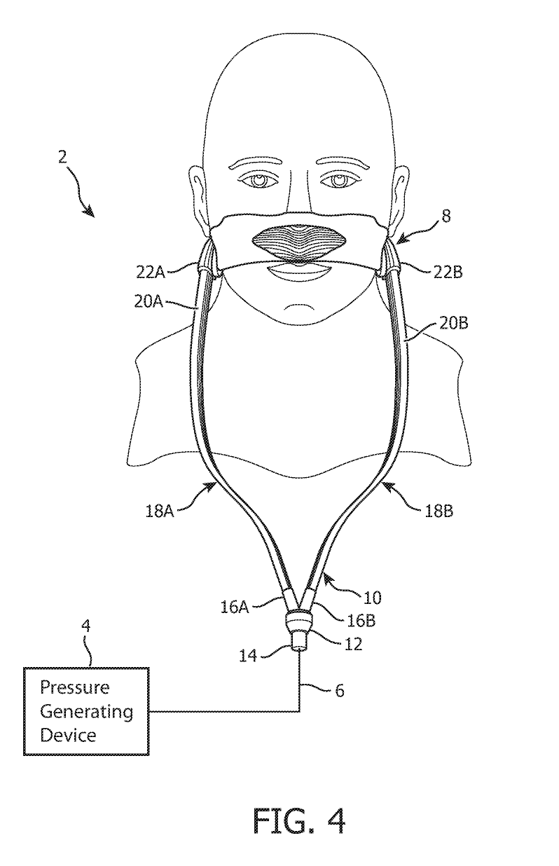

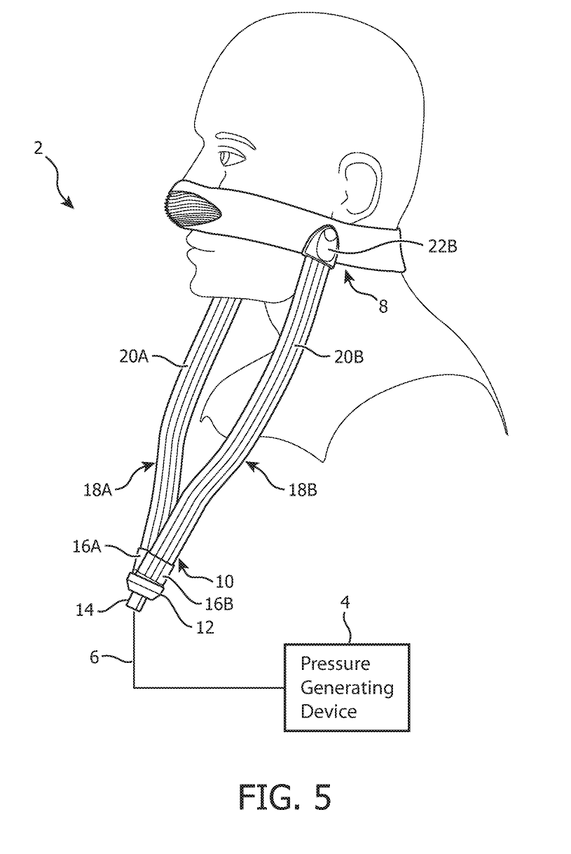

In operation, if the patient wishes to use patient sealing assembly 24 as a nasal/oral mask component, the patient will don patient sealing assembly 24 in a condition wherein adjustable sections 30, 36A and 36B are expanded such that the generally horizontal bellows, accordion or convoluted surface features 32 and 38 are spread apart. This configuration is shown in FIGS. 1-3. If, however, the patient wishes to use patient sealing assembly 24 as a nasal mask component, the patient will, before (or after) donning patient sealing assembly 24, cause adjustable sections 30, 36A and 36B to be contracted (in the direction of the arrows) such that the generally horizontal bellows, accordion or convoluted surface features 32 and 38 are not spread apart. This configuration is shown in FIGS. 4 and 5.

In alternative embodiments, bellows, accordion or convoluted surface features 32 may be replaced by an alternative adjustment features, such as an adjustable grid or mesh with articulating intersecting members, or a number of adjustable helix members.

Thus, patient interface device 8 as just described provides a mask component that may be converted from a nasal/oral mask to a nasal mask and back as desired to suit the needs of the patient.

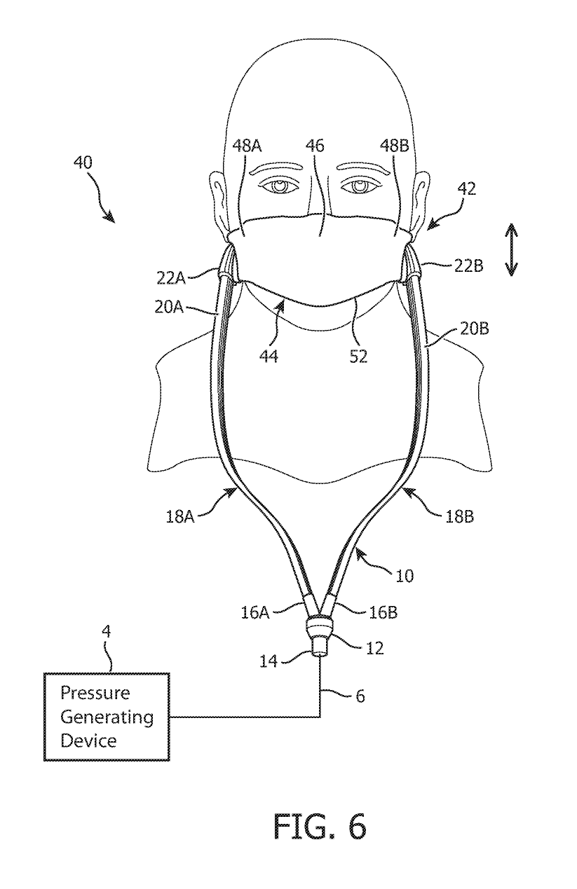

A system 40 adapted to provide a regimen of respiratory therapy to a patient according to an alternative exemplary embodiment is generally shown in FIGS. 6 (front view) and 7 (side view). System 40 is similar to system 2 described above, and like components are labeled with like reference numerals.

System 40, however, employs an alternative patient interface device 42 that includes an alternative patient sealing assembly 44 for facilitating the delivery of the flow of breathing gas from pressure generating device 4, delivery conduit 6 and fluid coupling member 10 to the airway of a patient. Patient sealing assembly 44 includes a central cushion portion 46, first and second side portions 48A and 48B, and rear portions 50 that are structured to extend around the rear of the patient's head/neck when patient interface device 42 is donned by the patient for securing patient interface device 42 to the head of the patient. Rear portions 50 may include any type of suitable attachment means for attaching to one another, such as, without limitation, a hook and loop fastening system or a magnetic fastening system.

In the exemplary embodiment, patient sealing assembly 44 is made of flexible, cushiony, elastomeric material, such as, without limitation, silicone, an appropriately soft thermoplastic elastomer, a closed cell foam, or any combination of such materials. Also in the exemplary embodiment, at least central cushion portion 46 and first and second side portions 48A and 48B are hollow/inflatable such that the flow of breathing gas can be received in first and second side portions 48A and 48B from first and second mask delivery members 18A and 18B, and then delivered to central cushion portion 46 for subsequent delivery to the airways of the patient.

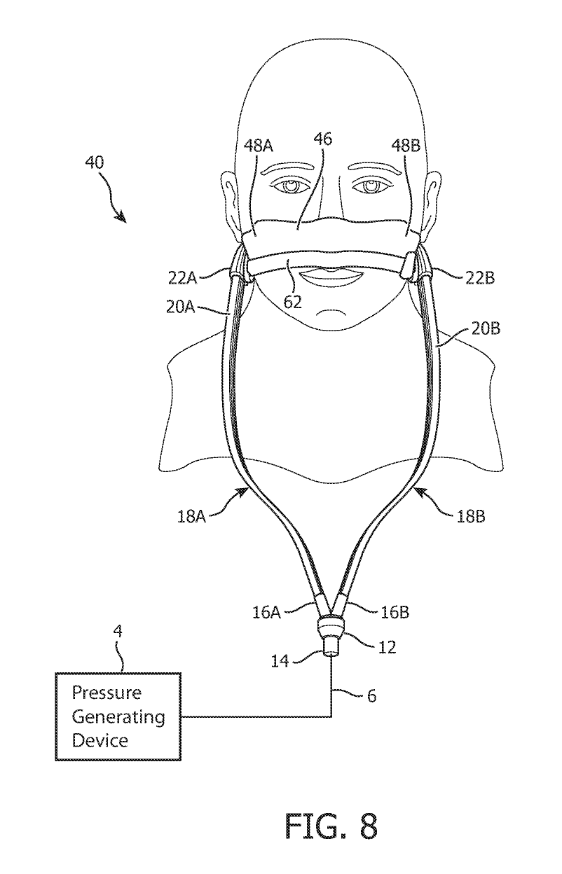

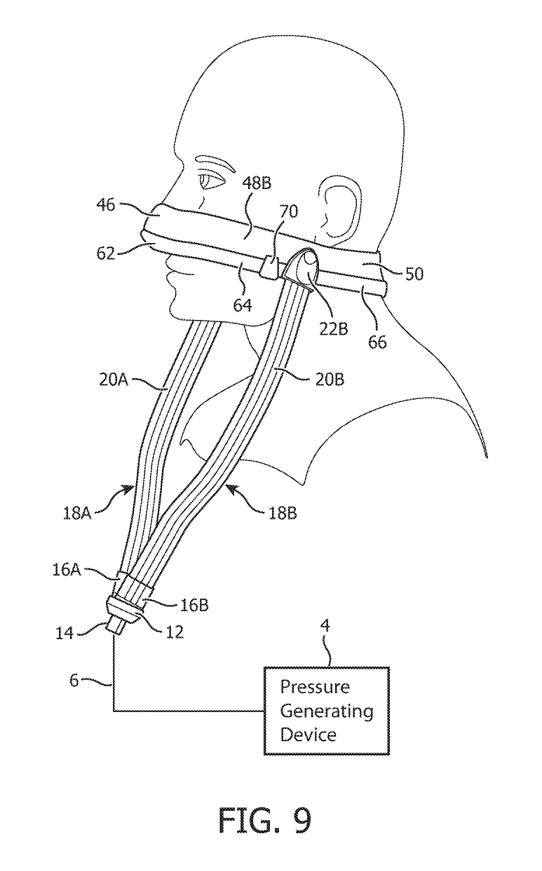

In addition, in the present embodiment, the bottom edges 52, 54 and 56 of central cushion portion 46, first and second side portions 48A and 48B, and rear portions 50 are structured and configured such that they may be selectively rolled up (on the inside or the outside of patient sealing assembly 44; outside being shown in the FIGS.) to form rolled sections 62, 64 and 66 as shown in FIGS. 8 and 9. The selective rolling of bottom edges 52, 54 and 56 to form rolled sections 62, 64 and 66 allows the size of central cushion portion 46 to be selectively adjusted in a direction that is generally perpendicular to the longitudinal axis of patient sealing assembly 44 as shown by the arrows in FIG. 6.

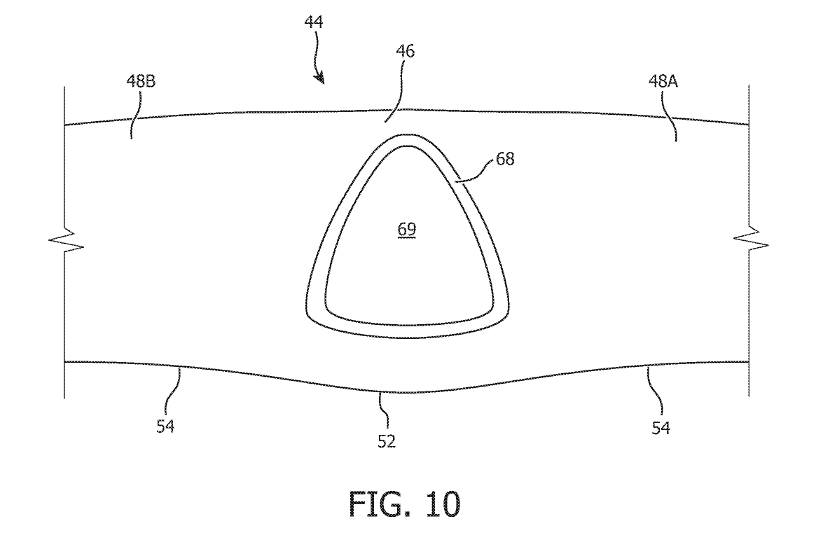

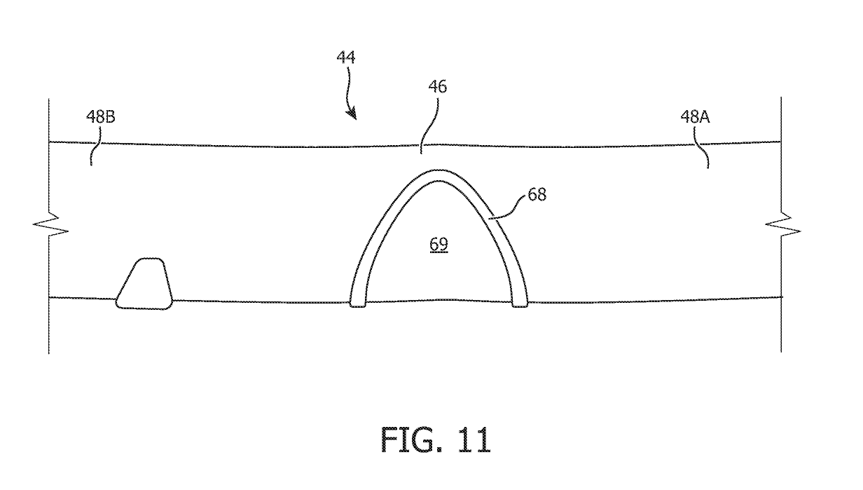

FIGS. 10 and 11 are partial rear elevational views of patient sealing assembly 44. As seen in FIGS. 10 and 11, the rear side of central cushion portion 46 of patient sealing assembly 44 further includes a generally triangularly shaped sealing surface 68 defining a fluid delivery opening 69. In the configuration shown in FIG. 10, sealing surface 68 is sized and structured to sealingly surround the nose and mouth of the patient to allow patient sealing assembly 44 to function as a nasal/oral mask component. Alternatively, in the configuration shown in FIG. 11, sealing surface 68 is sized and structured to sealingly surround just the nose of the patient to allow patient sealing assembly 44 to function as a nasal mask component.

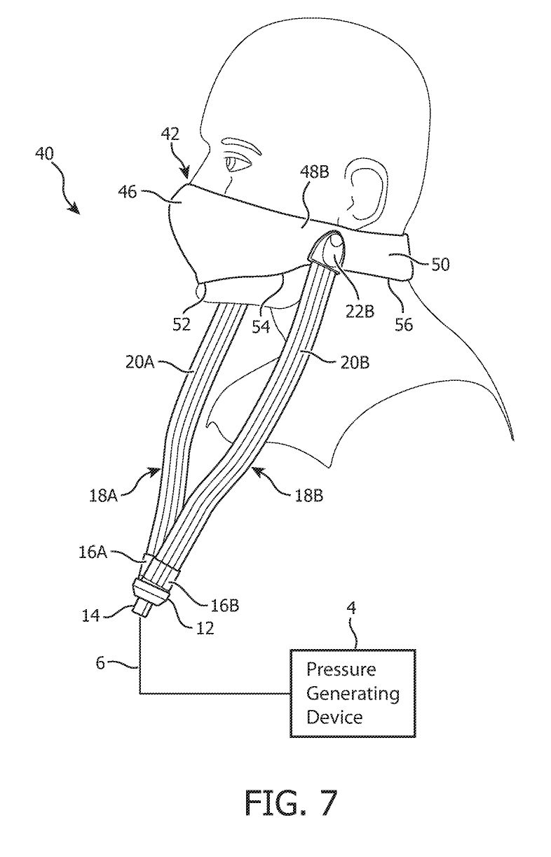

In operation, if the patient wishes to use patient sealing assembly 44 as a nasal/oral mask component, the patient will don patient sealing assembly 44 in a condition wherein bottom edges 52, 54 and 56 of central cushion portion 46, first and second side portions 48A and 48B are not rolled up. This configuration is shown in FIGS. 6 and 7. If, however, the patient wishes to use patient sealing assembly 44 as a nasal mask component, the patient will, before (or after) donning patient sealing assembly 44, roll up bottom edges 52, 54 and 56 of central cushion portion 46, first and second side portions 48A and 48B, and rear portions 50 to form rolled sections 62, 64 and 66. This configuration is shown in FIGS. 8 and 9. In addition, in the exemplary embodiment, a mechanism is provided for securing rolled sections 62, 64 and 66 so that they do not move after being set to the desired location. In the illustrated embodiment, this mechanism is a number of clip members 70 (e.g., made of a material such as silicone or plastic) that are attached to the bottom of patient sealing assembly 44 at one or more locations. Alternatively, the securing mechanism may be adhesive or some other fastening mechanism.

In a further alternative embodiment, the top edges of central cushion portion 46, first and second side portions 48A and 48B, and rear portions 50 may be structured and configured such that they may be selectively rolled up to form corresponding rolled sections, either in addition to or in place of rolled sections 62, 64 and 66.

Thus, patient interface device 44, as just described, like patient interface device 8, provides a mask component that may be converted from a nasal/oral mask to a nasal mask and back as desired to suit the needs of the patient.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A patient interface device, comprising:
a fluid coupling member structured to be coupled to a source of breathing gas;
a patient sealing assembly structured to be coupled to the fluid coupling member, the patient sealing assembly including a central cushion portion, a first side portion and a second side portion, wherein the central cushion portion is structured to enable a size of the central cushion portion to be adjusted in a direction that is generally perpendicular to a longitudinal axis of the patient sealing assembly from a first size configured to provide a nasal/oral mask configured to cover a nose and mouth of a patient to a second size configured to provide a nasal mask configured to cover only the nose of the patient, wherein the central cushion portion includes an adjustable section having a plurality of selectively expandable and contractible adjustment features structured to allow the size of the central cushion portion to be selectively adjusted in the direction that is generally perpendicular to the longitudinal axis of the patient sealing assembly.

2. (canceled)

3. The patient sealing assembly according to claim 1, wherein the plurality of adjustment features comprise a plurality of bellows, accordion or convoluted surface features that extend in a direction that is generally parallel to the longitudinal axis of the patient sealing assembly, the plurality of bellows, accordion or convoluted surface features being structured to fold outwardly or inwardly to allow the size of the central cushion portion to be selectively adjusted.

4. The patient sealing assembly according to claim 3, wherein the plurality of adjustment features are provided on an outer surface of the central cushion portion.

5. The patient sealing assembly according to claim 4, wherein an inner surface of the central cushion portion includes:
(i) a sealing surface defining a fluid delivery opening,
(ii) a first adjustable section having a plurality of first adjustment features provided on a first side of the sealing surface, and
(iii) a second adjustable section having a plurality of second adjustment features provided on a second side of the sealing surface.

6. The patient sealing assembly according to claim 5, wherein the first adjustment features comprise a plurality of first bellows, accordion or convoluted surface features that extend in a direction that is generally parallel to the longitudinal axis of the patient sealing assembly, and wherein the second adjustment features comprise a plurality of second bellows, accordion or convoluted surface features that extend in a direction that is generally parallel to the longitudinal axis of the patient sealing assembly

7. The patient sealing assembly according to claim 4, wherein the central cushion portion, the first side portion and the second side portion are hollow and are structured to receive the breathing gas from the fluid coupling member.

8. The patient sealing assembly according to claim 1, wherein the fluid coupling member includes an input port, first and second output ports, a first delivery member coupled to the first output port and the first side portion of the patient sealing assembly and a second delivery member coupled to second output port and the first side portion of the patient sealing assembly.

9. The patient sealing assembly according to claim 8, wherein the first delivery member includes a first conduit member having a first coupling member attached to a distal end thereof, and wherein the second delivery member includes a second conduit member having a second coupling member attached to a distal end thereof.

10.-13. (canceled)

14. A method of adjusting a patient interface device, comprising:
providing a patient sealing assembly including a central cushion portion, a first side portion and a second side portion;
adjusting a size of the central cushion portion in a direction that is generally perpendicular to a longitudinal axis of the patient sealing assembly from a first size configured to provide a nasal/oral mask configured to cover a nose and mouth of a patient to a second size configured to provide a nasal mask configured to cover only the nose of the patient or from the second size to the first size, wherein the central cushion portion includes an adjustable section having a plurality of selectively expandable and contractible adjustment features, wherein the adjusting comprises expanding or contracting the adjustment features.

15. (canceled)

16. The method according to claim 14, wherein the plurality of adjustment features comprise a plurality of bellows, accordion or convoluted surface features that extend in a direction that is generally parallel to the longitudinal axis of the patient sealing assembly, the plurality of bellows, accordion or convoluted surface features being structured to fold outwardly or inwardly to allow the size of the central cushion portion to be selectively adjusted.

17. (canceled)

18. A patient interface device, comprising:
a fluid coupling member structured to be coupled to a source of breathing gas;
a patient sealing assembly structured to be coupled to the fluid coupling member, the patient sealing assembly including a central cushion portion, a first side portion and a second side portion, wherein the central cushion portion is structured to enable a size of the central cushion portion to be adjusted in a direction that is generally perpendicular to a longitudinal axis of the patient sealing assembly from a first size configured to provide a nasal/oral mask configured to cover a nose and mouth of a patient to a second size configured to provide a nasal mask configured to cover only the nose of the patient, wherein an edge of the central cushion portion and the first and second side portions is structured to be rolled up upon itself to create rolled sections to enable the size of the central cushion portion to be adjusted in the direction that is generally perpendicular to the longitudinal axis of the patient sealing assembly.

19. The patient sealing assembly according to claim 1, wherein the edge comprises a bottom edge of each of the central cushion portion and the first and second side portions.

20. The patient sealing assembly according to claim 1, further comprising means for securing the rolled sections in place.

21. The patient sealing assembly according to claim 3, wherein the means for securing comprises a clip member.

22. A method of adjusting a patient interface device, comprising:
providing a patient sealing assembly including a central cushion portion, a first side portion and a second side portion;
adjusting a size of the central cushion portion in a direction that is generally perpendicular to a longitudinal axis of the patient sealing assembly from a first size configured to provide a nasal/oral mask configured to cover a nose and mouth of a patient to a second size configured to provide a nasal mask configured to cover only the nose of the patient or from the second size to the first size, wherein an edge of the central cushion portion and the first and second side portions is structured to be rolled up upon itself to create rolled sections, and wherein the adjusting comprises rolling the edge up to create the rolled sections.
